(19) United States
(12) Patent Application Publication
(10) Pub. No.: US 2014/0284304 A1
(43) Pub. Date: Sep. 25, 2014

(54) METHOD OF FABRICATING TEST STRIP OF BIOLOGICAL FLUID

(71) Applicant: Ichia Technologies, Inc., Taoyuan (TW)

(72) Inventor: Ko-Ju CHEN, Taoyuan (TW)

(73) Assignee: Ichia Technologies, Inc., Taoyuan (TW)

(21) Appl. No.: 14/018,746

(22) Filed: Sep. 5, 2013

(30) Foreign Application Priority Data
Mar. 22, 2013 (TW) 102110208

(51) Int. Cl. G01N 33/66

(52) U.S. Cl. CPC G01N 33/66
USPC 216/20; 156/277

(57) ABSTRACT

The invention provides a method of fabricating test strips for measuring biological blood. The method comprises steps: preparing an insulating membrane material; printing a metallic circuit layer on the insulating membrane material by a metallic ionic ink; performing a surface treatment for the metallic circuit layer expose metallic ions of the metallic ionic ink; chemical plating for the exposed metallic ions of the metallic circuit layer to form an electrode section on the metallic circuit layer; providing a sensing reagent on the electrode section to form a sensing reagent layer; sequentially adhering an intermediate layer and a cover to the insulating membrane material to cover the metallic circuit layer and expose part of the metallic circuit layer; and cutting the insulating membrane material to form a plurality of insulating sheets. The test strip is provided to enhance the stability of resistance and the test accuracy.

### BACKGROUND OF THE INVENTION

1. Field of the Invention

The present invention relates to a test strip, and more in particular, to a method for fabricating test strip of measuring biological blood.

2. Related Art of the Invention

A blood glucose test is one of blood test items. One of the current technologies is proposed including steps of: blood is instilled on a test strip, the blood is sensed by a sensing reagent, a signal is transmitted to a blood glucose meter by a metallic circuit of the test strip, and blood glucose concentration of the blood is measured by the blood glucose meter.

A conventional test strip such as electrode type test strip for a biosensor includes abase body with a printed surface, an intermediate layer and a cover. The printed surface has a reactive membrane, two electrode membranes, a conductive electrode membrane and a reference electrode membrane. The conductive electrode membrane is disposed between two electrode membranes. The two electrode membranes connect with the reactive membrane at one end of each electrode membrane. The conductive electrode membrane connects to one of two electrode membranes at adjacent the reactive membrane. The reference electrode membrane is disposed and opposite to one of two electrode membranes at two sides of the printed surface. The reference electrode membrane extends to contact the reactive membrane at one end. The intermediate layer has an opening corresponding to the reactive membrane, and the cover has a test hole corresponding to the opening. The cover and the intermediate layer are disposed on the base body in sequence, and the intermediate layer is interposed between the base body and the cover and adhesive to all together.

The above-mentioned conductive layer (electrode) is fabricated by printing a conductive carbon powder and silver paste on the base body in a printing process. However, the conductive layer fabricated in a printing manner has poor test accuracy due to poor stability of resistance.

### SUMMARY OF THE INVENTION

It is an object of the invention to provide a test strip in a combination of printing manner and chemical plating such as metallic ion replacement or electroless plating to solve the above-described drawback of the conventional test strip. The metallic circuit layer is provided in a combination of printing manner and chemical plating to enhance the stability of resistance and the test accuracy.

In order to achieve the above-described object, the invention provides a method of fabricating test strips for measuring biological blood. The method comprises steps: preparing an insulating membrane material; printing a metallic circuit layer on the insulating membrane material by a metallic ionic ink; performing a surface treatment for the metallic circuit layer to expose metallic ions of the metallic ionic ink by treating the insulating membrane material printed with the metallic ionic ink; chemical plating for the exposed metallic ions of the metallic circuit layer to form an electrode section on the metallic circuit layer; providing a sensing reagent on the electrode section to form a sensing reagent layer; adhering an intermediate layer to the insulating membrane material to cover the metallic circuit layer and expose part of the metallic circuit layer, the intermediate layer having an opening to expose the sensing reagent layer and the electrode section; and adhering a cover to cover the intermediate layer and the insulating membrane material and expose part of the metallic circuit layer, the cover having two holes corresponding to the opening.

The insulating membrane material may be paper, timber, Acrylonitrile-Butadiene-Styrene (ABS), polycarbonate (PC), polyethylene terephthalate (PET), polyimide (PI), thermoplastic elastomer or polylactic acid plastic.

The surface treatment of the metallic circuit layer may be performed by baking or immersing into an acidic solution to melt or etch the resins disposed the surface of the metallic ionic ink so that the metallic ions of the metallic ionic ink may be exposed.

The chemical plating of the exposed metallic ions of the metallic circuit layer may be performed by metallic ion replacement or electroless plating to complete the resultant metallic circuit layer that includes an electrical connector, a metallic circuit connecting to the electrical connector and an electrode section connecting to an end of the metallic circuit. The metallic ion replacement or electroless plating is performed by immersing the exposed metallic ions into an active solution including metallic ions which are different to the exposed metallic ions to replace the metallic ions of the metallic ionic ink with the metallic ions of the active solution.

The amount and species of the sensing reagent are selected as required. The sensing reagent is applied on the electrode section to form a sensing reagent layer by spray marking.

The intermediate layer is adhered to the insulating membrane material to cover the metallic circuit and the electrode section of the metallic circuit layer and expose the electrical connector. The intermediate layer is shorter than the insulating membrane material. The intermediate layer is of insulating material.

After the cover is covered, the test strip exposes the electrical connector of the metallic circuit layer. The cover is shorter than the insulating membrane material. The cover is of insulating material.

The insulating membrane material is cut to form a plurality of insulating sheets. Each of the insulating sheets has the metallic circuit layer, the sensing reagent layer, the intermediate layer and the cover.

The resistance of the metallic circuit layer has a range from 0.01 Ω to 2 k Ω. The thickness of the metallic circuit layer has a range from 0.02 um to 30 um.

### DESCRIPTION OF DRAWINGS

FIG. 1 schematically shows a flow chart of fabricating test strips for measuring biological fluid of an embodiment of the invention.

FIGS. 2-3 schematically show cross-sectional views of a test strip by a part of steps of fabricating process of an embodiment of the invention.

FIG. 4 is a top view of FIG. 3.

FIGS. 5-7 schematically show top views of a test strip by a part of steps of fabricating process of an embodiment of the invention.

FIG. 8 is a schematic top view of a test strip for measuring biological fluid of the present invention.

FIG. 9 schematically shows a cross-sectional view of FIG. 8.

### DETAILED DESCRIPTION OF THE INVENTION

The present invention will be apparent from the following detailed description, which proceeds with reference to the accompanying drawings. The accompanying drawings are not meant to be construed in a limiting sense, which are only for reference and explanation.

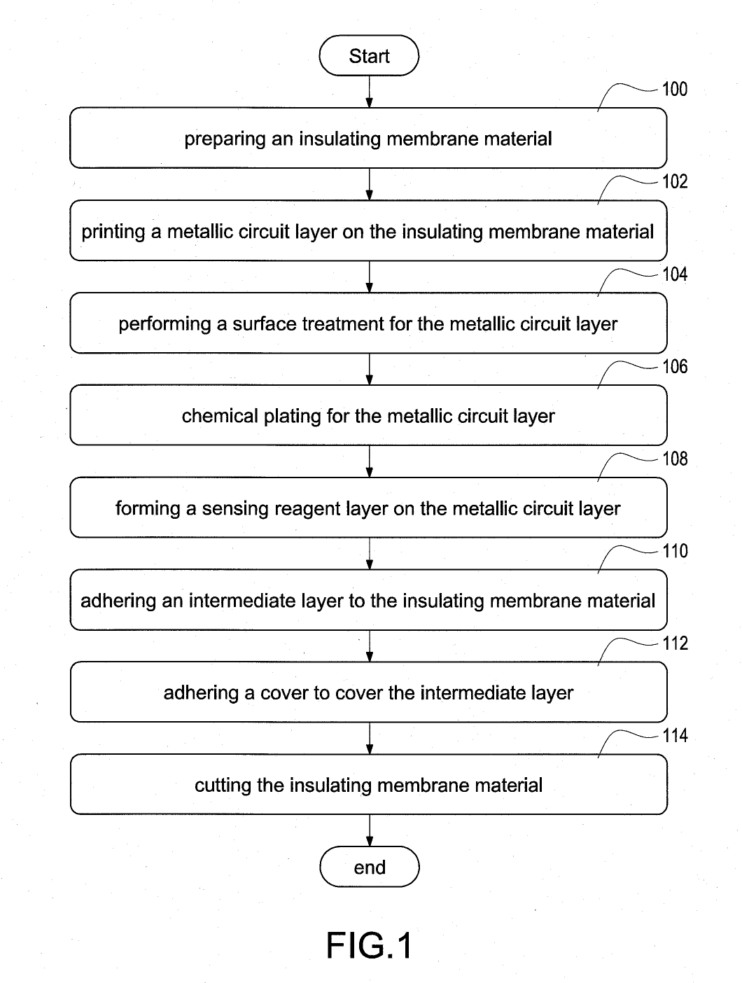

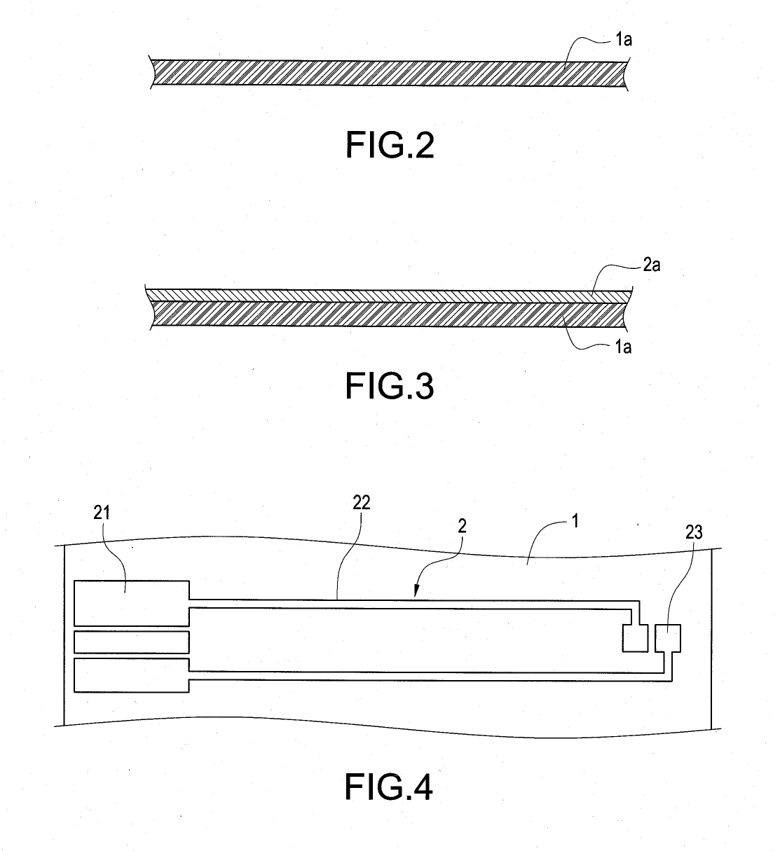

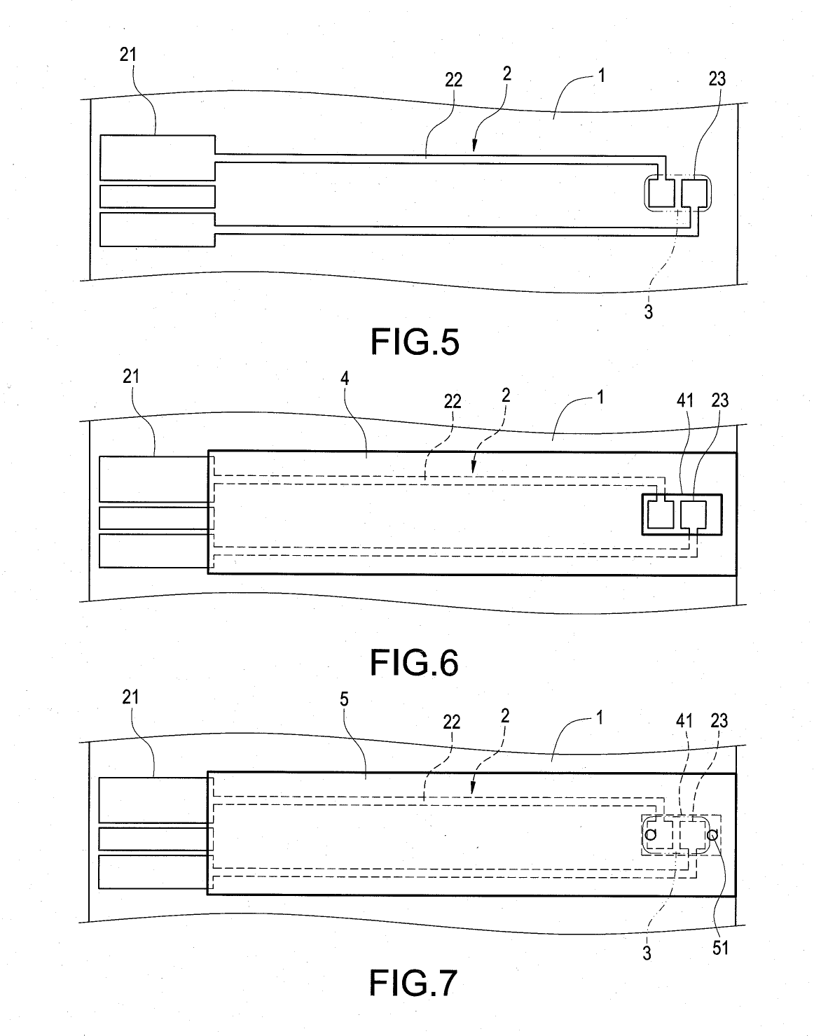

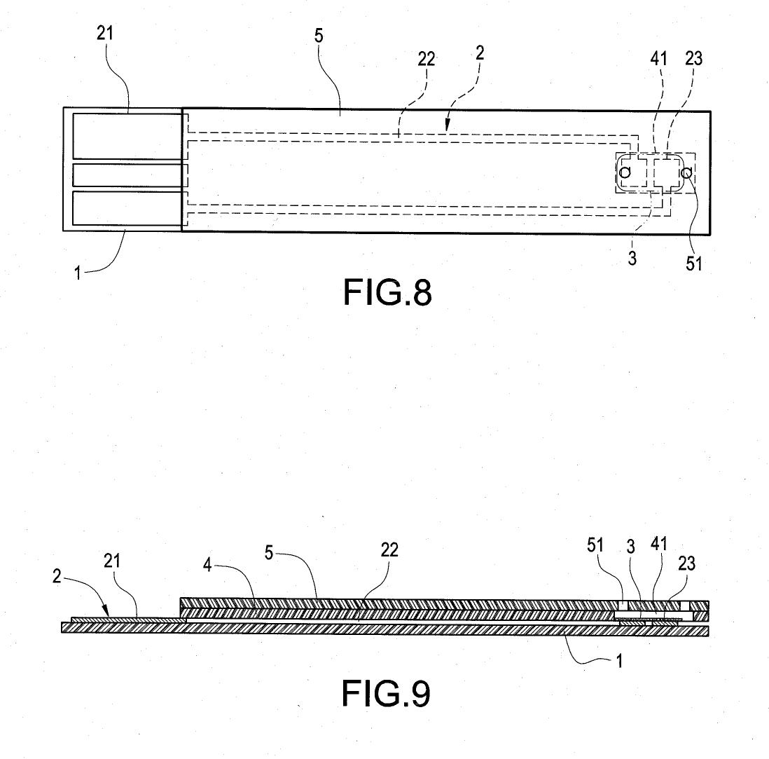

Please refer to FIG. 1. FIG. 1 schematically shows a flow chart of fabricating test strips for measuring biological fluid of an embodiment of the invention. Please also refer to FIGS. 2-9. FIGS. 2-3 schematically show cross-sectional views of a test strip by a part of steps of fabricating process of an embodiment of the invention. FIG. 4 is a top view of FIG. 3, FIGS. 5-8 schematically show top views of a test strip by a part of steps of fabricating process of an embodiment of the invention. FIG. 9 is a cross-sectional view of FIG. 8. As shown in drawings, the method of fabricating test strips for measuring biological fluid of the invention is provided. At first, in step 100, an insulating membrane material is prepared. In the embodiment, the insulating membrane material 1a may be paper, timber, Acrylonitrile-Butadiene-Styrene (ABS), polycarbonate (PC), polyethylene terephthalate (PET), polyimide (PI), thermoplastic elastomer or polylactic acid plastic, as shown in FIG. 2.

In step 102, a metallic circuit layer is printed on a surface of the insulating membrane material 1a with a metallic ionic ink 2a by screen printing, lithography, digital printing and ink jetting, as shown in FIG. 3.

In step 104, a surface treatment of the metallic circuit layer may be performed by baking the insulating membrane material 1a printed with the metallic ionic ink 2a or immersing the insulating membrane material 1a printed with the metallic ionic ink 2a into an acidic solution to melt or etch the resins disposed the surface of the metallic ionic ink 2a so that the metallic ions of the metallic ionic ink 2a may be exposed.

In step 106, the chemical plating of the exposed metallic ions of the metallic circuit layer may be performed by metallic ion replacement or electroless plating to complete the resultant metallic circuit layer 2 that includes an electrical connector 21, a metallic circuit 22 connecting to the electrical connector 21 and an electrode section 23 connecting to an end of the metallic circuit 22 as shown in FIG. 4. The metallic ion replacement or electroless plating is performed by immersing the exposed metallic ions into an active solution including metallic ions of copper ions or nickel ions which are different to the exposed metallic ions to replace the metallic ions of the metallic ionic ink 2a with the metallic ions of the active solution.

Example 1

[CuSO4]aq+Fesolid→Cusolid+[FeSO4]Haq Cu+Pd2++→Cu2++Pd

Ni2++H2PO2−+H2O→Ni0+H2PO3−+2H+2Au++Ni→2Au+Ni2+

Example 2

[PdSO4]aq+Fesolid→Pdsolid+[FeSO4]Haq Ni2++H2PO2−+H2O→Ni0+H2PO3−+2H+2Au++Ni→2Au+Ni2+

In step 108, the sensing reagent is applied on the electrode section 23 of the metallic circuit layer 2 to form a sensing reagent layer 3 by spray marking, as shown in FIG. 5. The amount and species of the sensing reagent are selected as required.

In step 110, the intermediate layer 4 is adhered to the insulating membrane material la to cover the metallic circuit 22 and the electrode section 23 of the metallic circuit layer 22 and expose the electrical connector 21, the intermediate layer 4 having an opening 41 to expose the sensing reagent layer 3 and the electrode section 23, as shown in FIG. 6. In the embodiment, the intermediate layer 4 is shorter than the insulating membrane material 1a. The intermediate layer 4 is of insulating material.

In step 112, a cover 5 is covered on the intermediate layer 4, and then the test strip exposes the electrical connector 21 of the metallic circuit layer 2. The cover 5 has two holes 51 corresponding to the opening 41. The two holes 51 are provided for user to instill biological fluid, as shown in FIG. 7. In the embodiment, the cover 5 is shorter than the insulating membrane material 1a. The cover 5 is of insulating material.

In step 114, the insulating membrane material 1a is cut to form a plurality of insulating sheets 1. Each of the insulating sheets 1 has the metallic circuit layer 2, the sensing reagent layer 3, the intermediate layer 4 and the cover 5, as shown in FIGS. 8 and 9.

The test strip is determined after the fabrication has been completed. The resistance of the metallic circuit layer 2 has a range from 0.01 Ω to 2 k Ω. The thickness of the metallic circuit layer 2 has a range from 0.02 um to 30 um.

Also, as blood flows into inside of the test strip through the two holes 51, and the sensing reagent layer 3 sensing the blood, the resistance can keep stable during signal transmission because the metallic circuit layer 2 is fabricated by printing manner and chemical plating such as metallic ion replacement and electroless plating. Therefore, it can make glucose reaction much stable so that glucose meter can read accurate readings.

Please refer to FIGS. 8 and 9. FIG. 8 is a schematic top view of test strip for measuring biological fluid of the present invention. FIG. 9 is a schematic cross-sectional view of test strip for measuring biological fluid of the present invention. As shown in FIGS. 8 and 9, the test strip for measuring biological fluid of the present invention comprises an insulating sheet 1, a metallic circuit layer 2, a sensing reagent layer 3, an intermediate layer 4 and a cover 5.

The insulating sheet 1 may be paper, timber, Acrylonitrile-Butadiene—Styrene (ABS), polycarbonate (PC), a mixture of ABS and PC, polyethylene terephthalate (PET), polyimide (PI), thermoplastic elastomer or polylactic acid plastic.

The metallic circuit layer 2 may be disposed on the insulating sheet 1. The metallic circuit layer 2 includes an electrical connector 21, a metallic circuit 22 connecting to the electrical connector 21 and an electrode section 23 connecting to an end of the metallic circuit 22.

The sensing reagent layer 3 is disposed on the electrode section 23 of the metallic circuit layer 2. In the embodiment, the amount and species of the sensing reagent are selected as required.

The intermediate layer 4 is covered to the insulating sheet 1 and the metallic circuit 22 of the metallic circuit layer 2, and the intermediate layer 4 has an opening 41 to expose the sensing reagent layer 3 and the electrode section 23. The intermediate layer 4 is shorter than the insulating sheet 1. In the embodiment, the intermediate layer 4 is of insulating material.

The cover 5 is covered on the insulating sheet 1 and the intermediate layer 4. After the cover 5 is covered, the test strip only exposes the electrical connector 21. The cover 5 has two holes 51 corresponding to the opening 41. The two holes 51 are provided for a user to instill biological fluid. In the embodiment, the cover 5 is shorter than the insulating sheet 1. The cover 5 is of insulating material.

The test strip is determined after the fabrication has been completed. The resistance of the metallic circuit layer 2 has a range from 0.01 Ω to 2 k Ω. The thickness of the metallic circuit layer 2 has a range from 0.02 um to 30 um.

Also, as blood flows into inside of the test strip through the two holes 51, and the sensing reagent layer 3 senses the blood, the resistance can keep stable during signal transmission because the metallic circuit layer 2 is fabricated by printing manner and chemical plating such as metallic ion replacement and electroless plating. Therefore, it can make glucose reaction much stable so that glucose meter can read accurate readings.

Moreover, in case that the metallic circuit layer 2 has features of anti-oxidation and sensing, the step of film plating may be omitted in the fabrication of the test strip, and thus the fabrication of the test strip is simple and save working hours.

While the invention is described in by way of examples and in terms of preferred embodiments, it is to be understood that the invention is not limited thereto. On the contrary, the aim is to cover all modifications, alternatives and equivalents falling within the spirit and scope of the invention as defined by the appended claims.

What is claimed is:

1. A method of fabricating test strips for measuring biological blood comprising steps of:
(a) preparing an insulating membrane material;
(b) printing a metallic circuit layer on the insulating membrane material by a metallic ionic ink;
(c) performing a surface treatment for the metallic circuit layer to expose metallic ions of the metallic ionic ink by treating the insulating membrane material printed with the metallic ionic ink;
(d) performing a chemical plating for the exposed metallic ions of the metallic circuit layer to form an electrode section on the metallic circuit layer;
(e) providing a sensing reagent on the electrode section to form a sensing reagent layer;
(f) adhering an intermediate layer to the insulating membrane material to cover the metallic circuit layer and expose part of the metallic circuit layer, the intermediate layer having an opening to expose the sensing reagent layer and the electrode section; and
(g) adhering a cover to cover the intermediate layer and the insulating membrane material and expose part of the metallic circuit layer, the cover having two holes corresponding to the opening.

2. The method of fabricating a test strip for measuring biological fluid according to claim 1, wherein the insulating membrane material of step (a) is paper, timber, Acrylonitrile-Butadiene—Styrene (ABS), polycarbonate (PC), a mixture of ABS and PC, polyethylene terephthalate (PET), polyimide (PI), thermoplastic elastomer or polylactic acid plastic.

3. The method of fabricating a test strip for measuring biological fluid according to claim 2, wherein the surface treatment of step (c) of the metallic circuit layer is performed by baking or immersing into an acidic solution to melt or etch resins disposed a surface of the metallic ionic ink and expose the metallic ions.

4. The method of fabricating a test strip for measuring biological fluid according to claim 3, wherein the chemical plating of the step (d) is performed by metallic ion replacement or electroless plating with immersing the exposed metallic ions into an active solution including metallic ions which are different to the exposed metallic ions to replace the metallic ions of the metallic ionic ink with the metallic ions of the active solution.

5. The method of fabricating a test strip for measuring biological fluid according to claim 4, wherein the metallic circuit layer includes an electrical connector, a metallic circuit connecting to the electrical connector and an electrode section connecting to an end of the metallic circuit.

6. The method of fabricating a test strip for measuring biological fluid according to claim 5, wherein forming a sensing reagent layer of the step (e) is performed by spray marking the sensing reagent on the electrode section to form a sensing reagent layer.

7. The method of fabricating a test strip for measuring biological fluid according to claim 6, wherein the intermediate layer of the step (f) is adhered to the insulating membrane material to cover the metallic circuit and the electrode section of the metallic circuit layer and expose the electrical connector.

8. The method of fabricating a test strip for measuring biological fluid according to claim 7, wherein the intermediate layer is shorter than the insulating membrane material and the intermediate layer is of insulating material.

9. The method of fabricating a test strip for measuring biological fluid according to claim 8, wherein a cover of the step (g) is covered on the intermediate layer to expose the electrical connector of the metallic circuit layer, the cover is shorter than the insulating membrane material and the cover is of insulating material.

10. The method of fabricating a test strip for measuring biological fluid according to claim 9, after the step (g) further comprising cutting the insulating membrane material to form a plurality of insulating sheets, each of the insulating sheets having the metallic circuit layer, the sensing reagent layer, the intermediate layer and the cover.

11. The method of fabricating a test strip for measuring biological fluid according to claim 10, wherein the resistance of the metallic circuit layer has a range from 0.01 Ω to 2 k Ω.

12. The method of fabricating a test strip for measuring biological fluid according to claim 11, wherein the thickness of the metallic circuit layer has a range from 0.02 um to 30 um.
